(19) United States
(12) Patent Application Publication
(10) Pub. No.: US 2007/0089198 A1
(43) Pub. Date: Apr. 19, 2007

(54) Methods for Splicing Plant Genes

(75) Inventors: Joseph Chappell, Lexington, KY (US); Mark A. Schoenbeck, Omaha, NE (US); Bryan T. Greenhagen, Lexington, KY (US)

Correspondence Address: CATALYST LAW GROUP, APC, 9710 SCRANTON ROAD, SUITE S-170, SAN DIEGO, CA, 92121, US

(73) Assignee: University of Kentucky Research Foundation, Lexington, KY (US)

(21) Appl. No.: 11/610,733

(22) Filed: Dec. 14, 2006

Related U.S. Application Data
(62) Division of application No. 10/633,590, filed on Aug. 5, 2003, now abandoned.
(60) Provisional application No. 60/400,682, filed on Aug. 5, 2002.

(51) Int. Cl. A01H 1/00; C12N 15/82

(52) U.S. Cl. 800/280; 800/294

(57) ABSTRACT

A method for the recovery of full-length cDNAs for plant genes containing introns is described. The method utilizes Agrobacterium-mediated transient expression coupled with an RT-PCR assay to facilitate expression cloning of processed transcripts. Subsequent expression of intron-less cDNAs in a suitable prokaryotic or eucaryotic host provides for direction functional testing of the encoded gene product.

### RELATED APPLICATIONS

This application is a divisional of, and claims priority from, U.S. Utility patent application Ser. No. 10/633,590, filed Aug. 5, 2003, the entire disclosure of which is incorporated by reference herein.

### FIELD OF THE INVENTION

This invention relates to methods for generating intron-less plant cDNA. More particularly, the invention relates to methods for generating intron-less plant cDNAs involving transient expression, followed by agroinfiltration and reverse transcriptase-polymerase chain reaction (RT-PCR).

### BACKGROUND OF THE INVENTION

The increasing availability of data from plant genome projects has accelerated the rate of plant gene discovery and the need for functional analysis of these genes. Complimenting the analysis of gene mutants, studies employing heterologous expression in microbial systems provide a tractable means for demonstrating gene function. However, one constraint to microbial expression of plant genes is that yeast and bacterial systems lack the means to remove plant introns.

Splicing of RNA transcripts in plant cells comprises several distinct steps, reviewed by Brown and Simpson ((1998) Annu. Rev. Plant Physiol. Plant Mol. Biol. 49, 77-95) during which a ribonucleoprotein complex, called the spliceosome, is assembled. These steps proceed via the recognition of introns by trans-acting ribonucleoprotein factors, the definition of 5′ and 3′ intron splice junctions, the cleavage of the 5′ junction and formation of the “lariat” structure at a nucleotide site within the intron, and finally the cleavage at the 3′ junction and ligation of neighboring exons. Conserved sequence features are posited to interact with the RNA component of snRNPs to distinguish intron-splice junctions, though the absolute sequence requirements remain to be established (Brown and Simpson, 1998). Several studies have been undertaken with the goal of identifying sequences required for 5′ and 3′ splice site definition, either by the comparison of gene sequences (Brown and Simpson, (1996) Plant Mol. Biol. 32, 531-535), or by the experimental manipulation of intron or exon sequences to observe the effects on splicing function (Baynton et al., (1996) Plant J. 10, 703-711.; Egoavil et al., (1997) Plant J. 12, 971-980; Latijnhouwers et al, (1999) Plant Mol. Biol. 41, 637-644; McCullough and Schuler, (1997) Nucleic Acids Res. 25, 1071-1077). Using these data, algorithms have been developed to identify exon/intron boundaries (Brendel and Kleffe, (1998) Nucleic Acids Res. 26, 4748-4757; Hebsgaard et al., (1996) Nucleic Acids Res. 24, 3439-3452) for the purpose of predicting gene structure. Similarly, experiments have been undertaken to uncover internal sequences that define the intron branch point needed for lariat formation (Liu and Filipowicz, (1996) Plant J. 9, 381-389; Simpson et al., 1996). Equally important, plant genes are not accurately processed by other eukaryotic cells systems. For example, Sarmah et al. ((2002) Biochem. Biophys. Res. Commun. 293, 1209-16) examined the utility of Schizosaccharomyces pombe as an expression system for unprocessed plant genes using the AmA1 gene of Amaranthus hypocondriacus. Premature termination for a majority of transcripts was observed and intron splicing occurred at both genuine and aberrant sites (Sarmah et al., 2002), owing most probably to the differences in cis- and trans-acting splicing determinants between plants and yeast.

As a result, studies of plant gene function must begin with recovery or generation of intron-less clones. Spliced cDNAs can be recovered by RT-PCR or by cDNA library screening, but in either case knowledge of tissue specific expression is required. Alternatively, introns may be removed through in vitro manipulations, but the steps involved are time consuming and prone to introducing other alterations in the native DNA sequencing, and thus produce mutations in the encoded polypeptide.

Thus, there is a need for a method of producing intron-less plant cDNAs that does not require excessive manipulation of nucleic acids or knowledge of tissue specific expression of the gene of interest.

### SUMMARY OF THE INVENTION

In one aspect of the invention there is provided a method for producing intron-less plant cDNA. The method comprises the steps of (i) transforming Agrobacterium with a vector comprising a plant gene of interest operably linked to a promoter; (ii) infiltrating a leaf of a plant with the transformed Agrobacterium of (i) for a period of time to provide transient expression of the gene of interest;

(iii) isolating total RNA from the infiltrated leaf; (iv) performing RT-PCR using the total RNA as template; and (v) isolating intron-less plant cDNA corresponding to the gene of interest from the products of RT-PCR.

In another aspect of the invention there is provided a method for producing a recombinant plant gene in a bacterial or yeast cell. The method comprises (i) transforming Agrobacterium with a vector comprising a plant gene of interest operably linked to a promoter; (ii) infiltrating a leaf of a plant with the transformed Agrobacterium of (i) for a period of time to provide transient expression of the gene of interest; (iii) isolating total RNA from the infiltrated leaf; (iv) performing RT-PCR using the total RNA as template; (v) isolating intron-less plant cDNA corresponding to the gene of interest from the products of RT-PCR; and (vi) transforming bacteria or yeast cells with the intron-less plant cDNA and expressing the cDNA in the bacteria or yeast.

### BRIEF DESCRIPTION OF THE DRAWINGS

FIG. 1 is a diagram illustrating one embodiment of the method of the invention.

FIG. 2A is a diagram of a genomic GUS construct (CsMV:GUS+I) comprising the GUS gene with introns, operably linked to a cassava vein mosaic virus (CsMVA) promoter. FIG. 2B is a graph showing a time course of GUS expression, measured as MUG activity, in agroinfiltrated petunia leaves. FIG. 2C is a stained agarose gel showing the products of RT-PCR of total RNA isolated from infiltrated leaf after four days of culturing. Lanes 1 and 8, IBstEII marker; 2, positive control using unspliced construct with forward and reverse primers; 3, reverse transcribed RNA with forward and reverse primers; 4, RNA without reverse transcriptase, forward and reverse primers; 5, RT RNA, forward primer only; 6, RT RNA, reverse primer only; 7, no template, forward and reverse primers.

FIG. 3A is a diagram of intron-exon organization of EAS110, a putative sesquiterpene cyclase gene. 3B is a stained agarose gel showing RT-PCR products of RNA from agroinfiltrated Petunia: Lanes 1 and 8, IBstEII marker; 2, positive control using unspliced construct with forward and reverse primers; 3, reverse transcribed RNA with forward and reverse primers; 4, positive control using the EAS4 cDNA with forward and reverse primers; 5, RNA without reverse transcripase, forward and reverse primers; 6, RT RNA, forward primer only; 7, RT RNA, reverse primer only; 9, no template, forward and reverse primers.

### DETAILED DESCRIPTION OF THE INVENTION

The present invention provides a method for recovering an intron-less plant cDNA from a corresponding genomic sequence, requiring no a priori knowledge of gene expression patterns. Employing a combination of transient expression following agroinfiltration (Kapila. et al. (1997), Plant Sci. 122, 101-108) and RT-PCR, the present method enables recovery of a spliced cDNA from amplified genomic sequences without knowledge of gene expression patterns and without time consuming manipulation steps. The recovered cDNA can be used to transform bacteria or yeast.

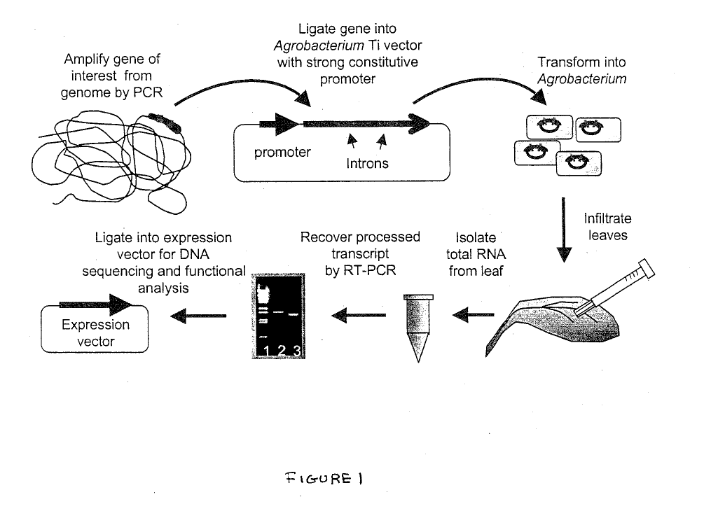

Prior studies have validated the utility of transient expression via agroinfiltration of leaves to study the biological activity of proteins in planta (Van der Hoorn, et al. (2000) Mol. Plant-Microbe Interact. 13, 439-446). Narasimhulu et al. ((1996) Plant Cell 8, 873-886) used RT-PCR of RNA from Agrobacterium-inoculated maize and tobacco cells to monitor the progress of T-DNA transfer. The present method has extended these prior art approaches to enable removal of introns from plant genes and recovery of full length cDNAs for subsequent expression in microbial systems. The method of the present invention, referred to herein as surrogate splicing, provides a rapid means for generating cDNA clones from plant genomic DNA sequences that can be expressed and analyzed in bacterial and yeast systems. This facilitates the functional analysis of predicted genes from plant genome sequencing projects by circumventing the need for information about gene expression patterns for in situ cDNA recovery. FIG. 1 illustrates the method of the invention.

In the present method intact genomic genes, which are comprised of introns and exons are amplified, preferably using the polymerase chain reaction (PCR) using genomic DNA as template. Genomic DNA is first isolated from the plant of interest using standard extraction procedures, e.g., See Sambrook et al. (1989).

Preferably the gene of interest is amplified, preferably using non-degenerate oligonucleotide primers specific for the gene. Typically the primers contain about 18 to about 45 nucleotides, however, the primers can be longer or shorter as needed. Preferably, the primers include restriction sites useful for cloning the amplified product into appropriate vectors.

The amplified genomic gene of interest is cloned into a suitable multiple cloning site of an Agrobacterium Ti-plasmid. Typical Ti-plasmids contain a strong constitutive or cell specific promoter and appropriately positioned T-DNA border sequences. For example, the cassava vein mosaic virus (CsVMV) promoter is a strong, constitutive promoter, which may be used in the present methods. T-DNA border sequences direct the transfer of the genomic gene and promoter sequences from the bacterium and promote their insertion into host plant DNA. The integrity of the construct may be determined by diagnostic PCR amplifications, restriction mapping, sequencing and the like, if desired.

The Ti-plasmid construct containing the genomic gene is then transformed into a suitable Agrobacterium, preferably an Agrobacterium tumefaciens strain, e.g., GV3850 using standard bacterial transformation protocols, e.g., triparental mating or electroporation. Transformed Agrobacterium carrying the construct are selected with appropriate medium, such as for example, medium containing an antibiotic to which the transformed bacteria are resistant, but to which non-transformed bacteria are sensitive. Kanamycin and rifampicin are examples of antibiotics typically used in selection for Ti-plasmid transformed Agrobacterium.

If desired, the presence of the Ti-plasmid construct in the selected Agrobacterium may be verified by standard PCR screening or by recovery and sequencing of the plasmid, for example.

The transformed Agrobacterium are grown in liquid culture to allow transient expression of the gene of interest. Typically, transformed bacteria are grown in nutrient rich broth, such as Luria broth, supplemented with appropriate selective antibiotics, such as kanamycin or rifampicin, for about 18 to about 36 hours at about room temperature, e.g. 28° C. The cells are then collected, preferably by centrifugation and washed to remove medium salts and antibiotics. The cells are resuspended in solution, such as sterile 10% sucrose solution and repelleted. The cells are then resuspended in a solution appropriate for infiltration, such as a sterile 10% sucrose solution. The final concentration of the cells is selected to maximize infiltration. Low Agrobacterium concentrations generally translate into significantly lower levels of GUS enzyme activity associated with the infiltration zones. Typically, the cells are concentrated to about A₆₀₀ 0.5±0.05.

Infiltration of leaves is carried out using standard protocols. For example, infiltration into the mesophyll tissues of the plant can be conducted by nicking the leaf surface, preferably the lower leaf surface, and infiltrating the transformed bacteria into the nicked tissue. Preferably the leaves used for infiltration are collected from rosettes of young plants, either monocotyledons or dicotyledons, such as young petunia plants, tobacco plants, Arabidopsis, and the like, although large leaves borne on stems may also be infiltrated. In a preferred embodiment, petunia leaves are used for agroinfiltration. Preferably, leaves from a dicotyledon are used to generate intron-less cDNAs of a gene derived from a dicotyledon, and monocotyledon leaves are used to generate intron-less cDNAs of a gene derived from a monocotyledon.

Prior to infiltration, the leaves are removed from the plant and washed to remove debris and increase the turgidity of the leaf to facilitate infiltration. The leaves are maintained in a moist environment until infiltration is complete. An applicator, such as a needleless syringe barrel typically used to apply the transformed bacteria to the prepared leaves. Infiltrated leaves are maintained in a moist environment, e.g., on damp filter paper for about two to about eight days, preferably about four days.

Alternatively, vacuum infiltration may be used. See, e.g., Kapila et al. (1997), Plant Science, 122:101-108. Briefly, leaves are placed in a suspension of transformed bacteria and a continuous vacuum in the range of about 1 to about 0.1 mbar is applied. During vacuum application, it is preferable that the flask containing the suspension is gently swirled to avoid trapping air bubbles. The vacuum is rapidly broken and the leaves rinsed and kept moist.

At about two to eight days following infiltration, and preferably about six days following infiltration, leaf discs are punched from the infiltrated leaves and the discs are pooled and stored at −80 degrees until time of RNA extraction. Total RNA is extracted from the leaf discs using standard protocols, such as using Invitrogen's Triazol reagent according to the manufacturer's instructions.

Total RNA is used to prepare cDNA using standard methods. Standard PCR is used to amplify cDNA and the gene of interest can be amplified using primers specific for the gene. The same primers used for amplification of the gene from the genomic template may be used to amplify cDNA or different gene-specific primers may be used to amplify the cDNA

The amplification product may be analyzed by agarose gel electrophoresis, for example, and compared to the amplification product of the full-length gene, which includes introns. A decrease in size indicates the removal of intron sequences.

The intron-less plant cDNA can be ligated into an appropriate expression vector, e.g., pET expression vectors, and transformed into or yeast for further studies. The transformed bacteria can be induced to express the intron-less plant cDNA using standard bacterial expression protocols.

### EXAMPLE 1

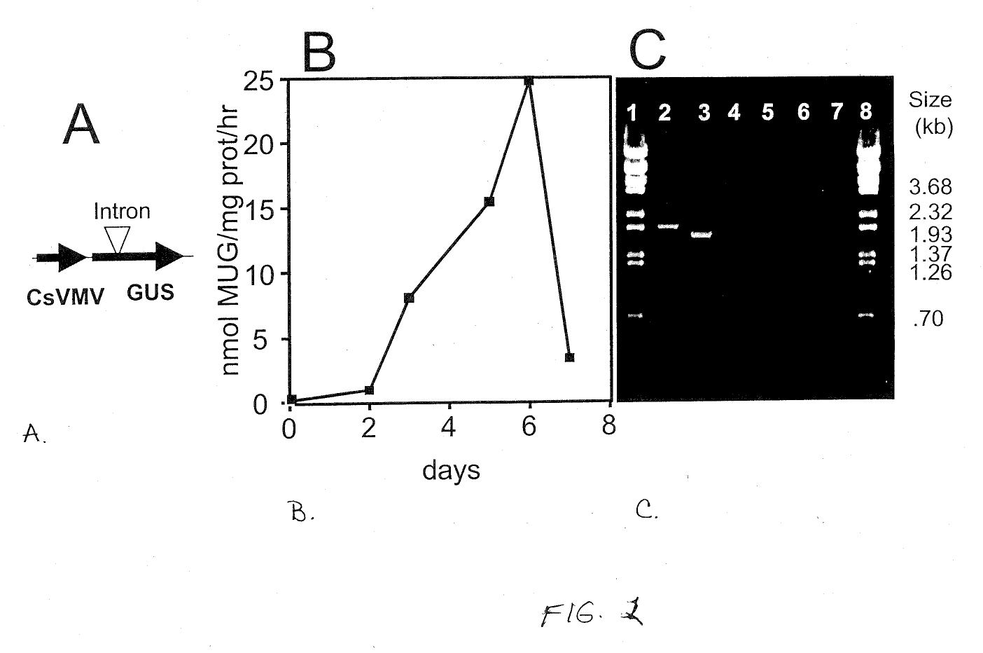

The time course for petunia mesophyll cells taking up and expressing T-DNA-borne transgenes following agroinfiltration was determined in leaf disks collected at daily intervals, and tested quantitatively for GUS enzyme activity (FIG. 2B). Detached petunia leaves were infiltrated with a suspension of A. tumefaciens carrying the intron-containing GUS gene (GUSI) driven by the cassava vein mosaic virus (CsVMV-GUSI) promoter, characterized for its ability to direct strong constitutive expression in leaf tissue (Verdaguer et al., 1998). GUS activity was absent or barely above background levels for the first two days after infiltration, then increased dramatically and almost linearly over the next four days. Maximum GUS activity was observed by six days post agroinfiltration and declined rapidly thereafter. The time course for transient expression of GUS activity in petunia is consistent with those previously reported for other plant species (Van der Hoorn et al., 2000). The absolute level of GUS activity in these transient expression studies was readily measured, and directly comparable to the levels observed in stable transgenic lines (Verdaguer et al., 1998).

### EXAMPLE 2

Recovery of a full-length cDNA from petunia leaf tissue agroinfiltrated with the GUSI gene was used to further assess the utility of this system for the generation of properly processed transcripts (FIG. 2b). Total RNA was isolated using a standard isolation procedure and 5 μg used for first-strand cDNA synthesis with an oligo-dT primer. An aliquot of the first-strand synthesis reaction was then used in combination with primers designed to bracket the start and stop codons of the GUS gene and containing convenient restriction sites for future insertion of the PCR fragments into suitable prokaryotic expression vectors. Single-primer, RNA-only, and template-less controls showed no amplification products (lanes 4-7), while the complete experimental reaction yielded a reaction product that was approximately 190 bp smaller than the positive control product amplified directly from the GUSI gene (compare lane 1 to lane 2). The amplification product of lane 6 was subsequently cloned into a commercially available vector harboring a 3′-thymidine over-hang (T/A cloning, Mezei and Storts, 1994), and sequenced. The sequence revealed that it was identical to the original GUS gene minus the 189-bp artificial intron (Vancanneyt et al., 1990) that had been properly removed at the 5′ (TAC/GTAA) and 3′ (GCAG/CT) splice sites.

### EXAMPLE 3

### Surrogate Splicing and Functional Analysis of a Putative Tobacco Sesquiterpene Synthase Gene

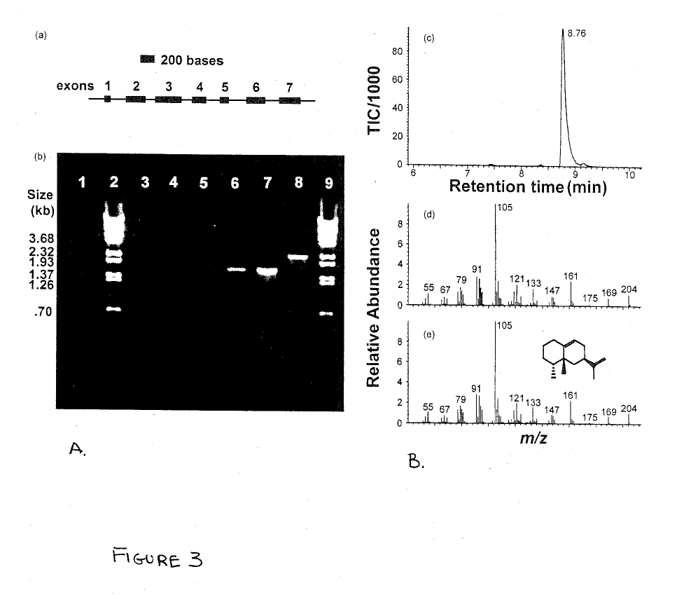

General applicability of surrogate splicing for functional analysis of an unknown gene containing several predicted introns was determined using a putative tobacco terpene synthase genomic clone referred to as g110 (FIG. 3). This genomic clone, along with approximately 30 other clones, was obtained when a Nicotiana tabacum cv. Xanthi genomic library was screened with a probe corresponding to the first 2 exons of the 5-epi-aristolochene synthase 4 gene (EAS4) (Facchini and Chappell, 1992). Sequence analysis of g110 revealed that it was 90% identical to the EAS4 gene at the nucleotide level (after insertion of 14 gaps to optimize for sequence alignment) and, like EAS4, was predicted to have seven exons punctuated by six introns (FIG. 3a). A single nucleotide deletion in the first exon at position +21, relative to the start ATG codon, resulted in a frame shift of the predicted g110-encoded protein. Whether this nucleotide deletion represents a genuine missense mutation in the tobacco genome, or an artifact of library construction and cloning did not bear directly on the evaluation of the splicing method, and was not resolved. Instead, the missing nucleotide was restored by PCR. The restored open reading frame resulted in a conceptual translation product 95% identical to that for EAS4.

Leaf disks of detached petunia leaves infiltrated with A. tumefaciens harboring the CsVMV-110 construct were collected after four days of incubation. RT-PCR using RNA from these leaves resulted in the amplification of a product (FIG. 3b, lane 3) significantly smaller than the positive size control of the g910 construct (2,327 bp, lane 2), but comparable in size to the amplification product from the authentic EAS4 cDNA (1,647 bp, lane 4). Control amplifications using single primers, RNA without reverse transcription, or no template, gave no comparable products (lanes 5, 6, 7, and 9). The product of the experimental reaction (lane 3) was T/A cloned and sequenced. The sequence of the recovered amplification product was identical to g110 except 6 intervening sequences had been removed accurately at the previously predicted intron-exon junctions.

The processed cDNA, termed RT110, was digested with suitable restriction enzymes and ligated into the pET28a expression vector (Novagen, Madison, Wis.), which adds a polyhistidine tag at the amino terminus of the expressed protein. Lysate of IPTG-induced E. coli bearing the pET28a-RT110 construct was recovered after sonication and centrifugation, and the histidine-tagged protein purified by chromatography over a nickel-affinity column to greater than 95% purity based on Coomassie blue staining of a denaturing polyacrylamide gel (Mathis et al., 1997). The purified protein was then incubated with farnesyl diphosphate, the substrate of known sesquiterpene synthases, under standard conditions (Rising et al., 2000). Gas chromatography showed a single major pentane-extractable product from this reaction (FIG. 3c). The mass spectrum of this major product (FIG. 3d) was identical to a genuine 5-epi-aristolochene standard (FIG. 3e), thus demonstrating that RT110 cDNA encodes for a functional 5-epi-aristolochene synthase activity.

The generally agreed upon consensus sequences for 5′ and 3′ splice sites in plants are AG/GTAAGT (62, 79, 100, 99, 70, 58, 49, 53% frequency of specified base) and TGCAG/G (64, 42, 95, 100, 100, 57% frequency of specified base), respectively (with/representing splice site) (Lorkovic et al., 2000). The six intervening sequences properly processed from the sesquiterpene synthase gene using the present method are consistent with these consensus sequences, including typical deviations within these sites. The composite 5′ and 3′ sites for the six splice sites are DR/GTRHGW and HXBAG/R. Hence, the present method accurately processes multiple introns having a typical range of splice-site sequences found in plant genes, which reflects the importance of other factors including other sequences and contextual positioning of these sequences for high fidelity processing (Lorkovic et al., 2000, Brown et al., 2002). Several studies have demonstrated how difficult gene identification based solely on sequence identity may be (Lehfeldt et al., 2000; Schoch et al., 2001). This is even more problematic when attempting to sort out enzymes like terpene synthases. These are enzymes that utilize a range of allylic diphosphate substrates (GPP, FPP and GGPP) and can catalyze quite distinct reactions from one another, although still maintaining a very high degree of sequence identity (Trapp and Croteau, 2001). The availability of the surrogate splicing method described herein provides yet another tool for the preparation of processed mRNAs that can be readily cloned and used in appropriate host systems. The surrogate splicing process, from infiltration to RT-PCR recovery of the cDNA, requires about four to about five days. The greater part of the effort before and after these steps entails the manipulation of genomic or cDNA clones in their respective vectors. The use of a recombination-based cloning system (Hartley et al., (2000) Genome Res. 10, 1788-1795), rather than the traditional endonuclease-based system may alleviate difficulties arising from incompatible restriction sites necessary for ligation steps, and a gene may be quickly moved into an Agrobacterium infection vector, processed, and the resultant cDNA moved into an expression vector with minimal manipulations.

### EXAMPLE 4

### Plant Materials and Agroinfiltration

Petunia×hybrida plants were started from commercially available seed. Plants were grown in the glasshouse, with supplemental light provided by sodium vapor lamps. Prior to experimentation, a population of plants was generated by crossing two parental lines chosen for broad rosette leaves suitable for infiltration. Seeds generated from this cross were collected and subsequently maintained as “population A”. Leaves for experimental infiltration were chosen on the basis of size with a 5 cm width minimum. Leaves were cut from plants using a razor blade and rinsed in tap water to remove any adhering debris. This brief submersion was also helpful to promote hydration of the leaf, as fully turgid leaves are preferable for infiltration. Immediately prior to infiltration, leaves were placed on dampened paper towels in plastic boxes.

Agrobacterium tumefaciens strain GV3850 was transformed by electroporation and maintained under kanamycin and rifampicin selection. Overnight cultures for infiltration were concentrated by centrifugation, resuspended in a 10% sucrose solution, re-concentrated, and finally resuspended in 10% sucrose to a final concentration of A₆₀₀=0.5±0.05. Petunia leaves were nicked on the lower leaf surface, and the bacterial suspension introduced using a needle-less syringe barrel. Infiltrated leaves were maintained in an open plastic container on wet paper towels for up to one week.

Leaf disks for GUS enzyme assays (Gallagher (1992) In Gus protocols: Using the GUS gene as a reporter of gene expression. Academic Press, Inc., pp. 47-59) (incorporated herein) or for RNA isolation were cut with a 1 cm diameter cork borer and stored at −80° C. until processing. RNA isolation was performed using Trizol according to the manufacturer's instructions (Invitrogen, Carlsbad, Calif.).

Molecular Manipulations

Intron-containing clones (intron-containing GUS, (Vancanneyt et al. (1990) Mol. Gen. Genet. 220, 245-250) or g110) were ligated into a Ti plasmid derived from pBI101 (Clontech), behind the cassava vein mosaic virus (CsVMV) promoter (Verdaguer, et al. (1998) Plant Mol. Biol., 37, 1055-1067). Reverse transcription of RNA to generate a first strand for PCR was performed as follows: Total RNA (5 μg) was combined with water and first strand primer, T₍₂₇₎V, 20 pmol, to a total volume of 12 μL. This mixture was heated to 70° C. for 10 min, then allowed to cool to room temperature, and the remaining reagents added: 10 units RNase inhibitor, 100 mM Tris HCl (pH 8.3), 40 mM KCl, 10 mM MgCl₂, 0.5 mM spermidine, 1.25 mM each dNTP, 4 mM sodium pyrophosphate, and 200 units of reverse transcriptase (Superscript II, Invitrogen) in a total volume of 20 μL. The reaction was incubated at 42° C. for 1 hr, then placed on ice. PCR reactions (50 μL) used 2 μL of first strand cDNA as template, and included 20 pmol of specific forward and reverse primers, 20 mM Tris-HCl (pH 8.4), 50 mM KCl, 1.5 mM MgCl₂, 80 μM each dNTP, and 1 unit Taq polymerase (Invitrogen Life Technologies, Carlsbad, Calif.). Taq polymerase was added after the first heating step. The amplification conditions were 95° C., 1 minute; 60° C., 1 minute; 72° C., 2 minute, for 30 cycles.

Primers for the amplification of the intron-containing GUS gene, for initial transgene construction and for RT-PCR recovery were (forward, incorporating a BamHI site) 5′-AAG GAT CCT ATG TTA CGT CCT GTA GM ACC-3′ (SEQ ID NO. 1), and (reverse, incorporating an SstI site) 5′-AAG AGC TCA TTG TTT GCC TCC CTG CT-3′ (SEQ ID NO. 2). Primers for the amplification, construction, and recovery of the g110 clone were (forward, incorporating a BamHI site) 5′-AAG GAT CCA TGG CCT CAG CAG CAG TTG CA-3′ (SEQ ID NO. 3), and (reverse, incorporating an SstI site) 5′-AAA GAG CTC GCA GCT CAA ATT TTG ATG GA-3′ (SEQ ID NO. 4). Restoration of the g110 reading frame employed a forward primer 5′-GGA TCC ATG GCC TCA GCA GCA GTT GCA AAC TAT GAA GAA GA-3 (SEQ ID NO. 5)′ which incorporates a deleted “A” (underlined) at position +21 relative to the start codon.

Amplification products were ligated into the pGEM-T Easy cloning vector (Promega, Madison, Wis.) for sequencing analysis. All the DNA sequencing reactions were performed using the BigDye™ Terminator Cycle sequencing kit (Perkin-Elmer, Wellesley, Mass.) with the sequences being read on an automated ABI Prism 310 Genetic Analyzer (Applied Biosystems, Foster City, Calif.). Bacterial expression of the sesquiterpene synthase used the pET vector system (Novagen, Madison, Wis.) according to Back and Chappell (1995).

Construction of pET28a-RT110

The RT-110 BamHI-SstI fragment was ligated into the pET-28a (Novagen, Madison, Wis.) multi-cloning site providing for an N-terminal hexahistidyl tag in expressed protein. This construct was transformed into BL21 (DE3) competent E. coli cells (Novagen) which were grown to OD₆₀₀=1.0, induced with 0.1 mM IPTG and incubated for five hours at 200 rpm and at 28° C. Cells were collected by centrifugation, the pellets were frozen overnight at −80° C., then sonicated three times for 30s, and lysate recovered after a 20 min centrifugation at 38,000×g. The lysate was filtered through a 0.45 micron filter and purified by Ni⁺ affinity chromatography as per Mathis et al (1997). Concentrated RT-110 protein was saved as a 50% glycerol stock at −80° C. Protein concentrations were estimated by the Bradford method using IgG as the standard (BioRad, Hercules, Calif.).

Analysis of Enzymatic Product

The activity and product profile for Ni⁺ affinity column purified RT-110 enzyme was determined by incubation with the substrate, farnesyl diphosphate, followed by partitioning of the reaction product into an organic solvent. Reactions contained 200 mM Tris-HCl pH 7.5, 40 mM MgCl₂ and 100 nM enzyme and were performed at 30° C. Quantitative assays for enzyme activity were performed in 50 μL and contained 0.2 uCi of 3H-FPP (NEN) and 40 μM FPP (Echelon Research Laboratories, Salt Lake City, Utah) with five to 30 minute incubations. Turnover rates of radioactive substrate to product were determined by counting aliquots of hexane extracts in a scintillation counter. For reaction product identification by GC-MS, reactions were scaled to 2.5 mL and contained 80 μM FPP with no radiolabel. Reactions were incubated 30 minutes, extracted twice with 2 mL of pentane, which was concentrated to approximately 50 μL for GC-MS analysis. GC-MS analysis was performed with an HP-GCDplus equipped with a DB-5 ms capillary column (30 m×0.25 mm, 0.25 μm phase thickness) and run with He as the carrier gas at 1 mL/min. Sample injections were splitless with an injection port temperature of 250° C. The oven was programmed to hold at 100° C. for one minute and then ramped to 270° C. at 8° C./min. The dominant product was compared to an authentic sample of 5-epi-aristolochene, kindly provided by Dr. Robert M. Coates, Department of Chemistry, University of Illinois.

We claim:

1. A method for producing a recombinant plant gene in a bacterial or yeast cell comprising:
(i) transforming Agrobacterium with a vector comprising a plant gene of interest operably linked to a promoter;
(ii) infiltrating a leaf of a plant with the transformed Agrobacterium of (i) for a period of time to provide transient expression of the gene of interest;
(iii) isolating total RNA from the infiltrated leaf;
(iv) performing RT-PCR using the total RNA as a template;
(v) isolating intron-less plant cDNA corresponding to the gene of interest from the products of RT-PCR; and
(vi) transforming bacteria or yeast cells with the intron-less plant cDNA and expressing the cDNA in the bacteria or yeast.

2. The method of claim 1, wherein the intron-less cDNA is full length cDNA.

3. The method of claim 1, wherein infiltration is carried out for a period of from about two days to about six days.

4. The method of claim 1, wherein the Agrobacterium is Agrobacterium tumefaciens.

5. The method of claim 1, wherein the promoter is a constitutive promoter.

6. The method of claim 1, wherein the promoter is a cassava vein mosaic virus promoter.

7. The method of claim 1, wherein the gene of interest comprises a plurality of introns.

8. The method of claim 1 further comprising isolating the expression product of the intron-less cDNA from the bacteria or yeast.

9. The method of claim 1, wherein the bacteria are E coli.

10. The method of claim 1, wherein the leaf is obtained from a monocotyledon.

11. The method of claim 1, wherein the leaf is obtained from a dicotyledon.
